(19) United States
(12) Patent Application Publication
(10) Pub. No.: US 2023/0407029 A1
(43) Pub. Date: Dec. 21, 2023

(54) COMPOSITION OF FOAM AND CUSHIONS THAT INCLUDE DIGESTERS, AND RELATED METHODS OF MANUFACTURE

(71) Applicant: SOFT-TEX GROUP, INC., Latham, NY (US)

(72) Inventors: Mark SMIDERLE, Midhurst (CA); Carl Hilton POWELL, JR., Amelia, OH (US)

(73) Assignee: SOFT-TEX GROUP, INC., Latham, NY (US)

(21) Appl. No.: 18/456,036

(22) Filed: Aug. 25, 2023

Related U.S. Application Data
(63) Continuation of application No. PCT/US2022/018063, filed on Feb. 27, 2022.
(60) Provisional application No. 63/154,317, filed on Feb. 26, 2021.

(51) Int. Cl. C08J 9/00; C08G 18/71; C08G 18/64; C12N 11/093; C12N 11/04; A47C 27/14

(52) U.S. Cl. CPC C08J 9/0014; C08G 18/71; C08G 18/6476; C08G 2230/00; C12N 11/04; A47C 27/14; C08J 2375/04; C12N 11/093

(57) ABSTRACT

Foam compositions, cushions, and related methods of manufacture are disclosed. The foam composition or cushion include (a) polymer that includes (i) a maximum of 60% polyol that includes multiple hydroxyl groups, and (ii) less than 50% isocyanate, (b) at least 40% filler suspended in the polymer, and (c) a plurality of digesters suspended in the polymer.

### CROSS-REFERENCE TO RELATED APPLICATIONS

This application is a by-pass continuation of PCT International Application No. PCT/US2022/018063, filed on Feb. 27, 2022, and entitled Composition of Foam and Cushions That Include Digesters, and Related Methods of Manufacture, which claims priority benefit of U.S. Provisional Patent Application No. 63/154,317, filed on Feb. 26, 2021, and entitled Composition of Foam and Cushions That Include Digesters, and Related Methods of Manufacture, the entire contents of which is hereby expressly incorporated herein by reference in its entirety.

### FIELD OF THE DISCLOSURE

The present disclosure generally relates to foam, and more particularly to compositions of foam and cushions that include digesters, and related methods of manufacture.

### BACKGROUND

Polyurethanes (PUs) are believed to represent almost 8% of all plastics produced and are believed to be the 6^{th} most-used polymer in the world (see Kemona, A., & Piotrowska, M. (2020). Polyurethane Recycling and Disposal: Methods and Prospects. Polymers, 12(8), 1752. https.//doi.org/10.3390/polym12081752). In 2015 the annual production of polyurethanes was estimated to be 17.9 million metric tons, and 26.4 metric tons are expected to be produced in 2021 (see Research and Markets, 2016). PUs are often used in foams or as coatings, adhesives, sealants, and elastomers. One type of foam is a rigid or hard foam that is typically a closed-cell foam. Another type of foam is flexible polyurethane foam (FPF), which is used in mattresses, cushions (e.g., pillows, automotive seats, car seats, shoe soles, etc.), other furniture, and various other products. FPF is a polymer that is produced during an exothermic reactions when alcohols (e.g., diols, triols, polyols) that contain two or more reactive hydroxyl (—OH) groups per molecule (R′—(OH)ₙ) react with an isocyanate (e.g., diisocyanates, polyisocyanates) that contains two or more isocyanate (—NCO) groups per molecule (R—(N═C═O)ₙ) in the presence of a catalyst or by activation with ultraviolet light. The group that is formed as a result of this reaction is known as a urethane linkage.

Industrially, the two aromatic and aliphatic polyisocyanates that are most common and form the basis of an estimated 95% of all polyurethanes include toluene diisocyanate or methylbenzene diisocyanate (collectively termed “TDI”), or methylene diphenyl diisocyanate or diphenylmethane diisocyanate (collectively termed “MDI”). TDI is often used in low-density flexible foams for cushions, whereas MDI is typically used to make rigid foams.

It is estimated that some polyurethanes will take up to 1,000 years to fully decompose. However, polyurethane remains an important component of many products that are used daily. Various efforts have been made to recycle or repurpose polyurethanes, and efforts have been made to reduce the environmental impact of polyurethanes. Despite existing methods and processes to address the environmental impact of polyurethane, more efforts and technological advancement are needed in order to reduce the environmental impact and speed up decomposition of polyurethanes.

While certain aspects of conventional technologies have been discussed to facilitate disclosure of the invention, Applicants in no way disclaim these technical aspects, and it is contemplated that the claimed invention may encompass one or more of the conventional technical aspects discussed herein.

In this specification, where a document, act or item of knowledge is referred to or discussed, this reference or discussion is not an admission that the document, act or item of knowledge or any combination thereof was, at the priority date, publicly available, known to the public, part of common general knowledge, or otherwise constitutes prior art under the applicable statutory provisions; or is known to be relevant to an attempt to solve any problem with which this specification is concerned.

### SUMMARY

Shortcomings of the prior art are overcome and additional advantages are provided through an improved foam and cushion composition and related methods of manufacture disclosed herein. In particular, foams such as TDI and MDI foams, cushions (e.g. mattresses, pillows, seat cushions, car seats, pads, mats, etc.), and their related methods of manufacture that are disclosed herein may help reduce the environmental impact of polyurethane. In particular, incorporating biodegradable fillers or suspended solids at high concentrations combined with the incorporation of digesters into foam and/or cushions can enhance biodegradability of products that incorporate polyurethanes. It is also contemplated that the improved foam and cushion compositions and related methods may prove useful in addressing other problems and deficiencies in a number of technical area. Therefore, the disclosed foam compositions, cushion compositions, and related methods of manufacture should not necessarily be construed as limited to addressing any of the particular problems or deficiencies discussed herein.

Certain embodiments of the presently disclosed foam compositions, cushion compositions, and methods of manufacture have several features, no single one of which is solely responsible for their desirable attributes. Without limiting the scope of the foam compositions, cushions, and methods of manufacture as defined by the claims that follow, their more prominent features will now be discussed briefly. After considering this discussion, and particularly after reading the section of this specification entitled “Detailed Description,” one will understand how the features of the various embodiments disclosed herein provide a number of advantages over the current state of the art.

In one aspect, disclosed herein is a foam composition that includes a polymer, at least 40% filler suspended in the polymer, and a plurality of digesters suspended in the polymer. The polymer further includes a maximum of 60% polyol, which includes multiple hydroxyl groups, and less than 50% isocyanate.

In another aspect is a cushion composition that includes a polymer, at least 40% filler suspended in the polymer, and a plurality of digesters suspended in the polymer. The polymer further includes a maximum of 60% polyol, which includes multiple hydroxyl groups, and less than 50% isocyanate.

In another aspect is a method of manufacture that includes producing a foam composition or cushion that includes a polymer, at least 40% filler suspended in the polymer, and a plurality of digesters suspended in the polymer. The polymer further includes a maximum of 60% polyol, which includes multiple hydroxyl groups, and less than 50% isocyanate.

These and other features and advantages of the disclosure and inventions will become apparent from the following detailed description of the various aspects of the invention taken in conjunction with the appended claims and the accompanying drawings.

### BRIEF DESCRIPTION OF THE DRAWINGS

Aspects described herein are particularly pointed out and distinctly claimed as examples in the claims at the conclusion of the specification. The foregoing and other objects, features, and advantages of the disclosure are apparent from the following detailed description taken in conjunction with the accompanying drawings in which:

FIG. 1 depicts an elevational perspective view of an exemplary foam slab, according to the present disclosure;

FIG. 2A depicts a magnified side view of an exemplary foam slab, according to the present disclosure;

FIG. 2B depicts a magnified side view of the exemplary foam slab of FIG. 2A having a greater concentration of suspended solids, according to the present disclosure;

FIG. 2C depicts a magnified side view of the exemplary foam slab of FIG. 2B having a plurality of digesters suspended in the polymer;

FIG. 3 depicts an elevational perspective view of an exemplary cushion, according to the present disclosure; and

FIG. 4 depicts an elevational perspective view of another exemplary cushion, according to the present disclosure.

### DETAILED DESCRIPTION

Aspects of the present disclosure and certain features, advantages, and details thereof, are explained more fully below with reference to the non-limiting embodiments illustrated in the accompanying drawings. Descriptions of well-known materials, fabrication tools, processing techniques, etc., are omitted so as to not unnecessarily obscure the details of the inventions. It should be understood, however, that the detailed description and the specific example(s), while indicating embodiments of inventions of the present disclosure, are given by way of illustration only, and are not by way of limitation. Various substitutions, modifications, additions and/or arrangements within the spirit and/or scope of the underlying inventive concepts will be apparent to those skilled in the art from this disclosure.

Approximating language, as used herein throughout disclosure, may be applied to modify any quantitative representation that could permissibly vary without resulting in a change in the basic function to which it is related. Accordingly, a value modified by a term or terms, such as “about” or “substantially,” is not limited to the precise value specified.

Described herein are compositions of foam and cushions that have less polyurethane such that disposal of the foam and/or cushion disclosed herein has less of an environmental impact than disposal of existing compositions of foam and cushions that include foam. In one aspect, disclosed herein is a foam composition and/or a cushion that includes a polymer and at least 40% filler suspended in the polymer. The 40% filler reduces the amount of polymer (e.g., polyurethane) in the foam and/or cushion, thereby decreasing the environmental impact that would result from disposal of the foam and/or cushion when compared to existing foam and/or cushions. Further, the digesters (e.g., fungus to metabolize urethane, microbes that excretes acid, and/or algae for breaking down biofilm, etc.) suspended in the polymer can catalyze decomposition of the polymer of the foam composition and/or cushion. The polymer further includes a maximum of 60% polyol, which includes multiple hydroxyl groups, and less than 50% isocyanate. The isocyanate could include, for example, TDI or MDI. In forming the foam composition and/or cushion, the isocyanate and polyol may be combined with a blowing agent (e.g., water). The polyol and isocyanate may form a foam network, and the blowing agent may react with the isocyanate to produce CO₂ gas and generate heat.

Polyol may be, for example, within the range of about 2% to about 60% weight of the polymer of the foam composition or of the cushion and may be used in the formation of polyurethane and/or polyisocyanurate-based polymers. The polyol may be, for example one to six polyether and/or polyester polyols derived from various products. For instance, the polyol may be derived from petroleum, animal by-products, or natural oils. Isocyanate may be, for example, within the range of about 1% to about 50% weight of the polymer of the foam composition or of the cushion. The isocyanate may be used in the formation of polyurethane and/or polyisocyanurate-based polymers. The isocyanate index, which is the ratio of isocyanate equivalents to polyol equivalents in the formation of polyurethane and/or polyisocyanurate-based polymers, may be within the range of about 0.6-1.4. Further, the blowing agent may include one to five hydrocarbon-based liquid and/or gas compounds that may be used to expand the polyurethane and/or polyisocyanurate-based polymers. The blowing agent may be, for example, within the range of about 0% to about 6% weight of the polymer of the foam composition or of the cushion.

Other ancillary chemicals may also be included such as blowing catalysts, surfactant, stabilizing additives, auxiliary agents, and/or gelation catalysts. Blowing catalysts (e.g., one to four amine and/or metal containing liquid) can catalyze the reaction (e.g., polyurethane and/or polyisocyanurate reactions) with the blowing agent and lead to polyurea structures. Blowing catalysts may be in the range of about 0% to about 3% weight of the polymer. Surfactant (e.g., one to three siloxane containing liquids) may be, for example, within the range of about 0% to about 3% weight of the polymer of the foam composition or of the cushion and may be used to modify the polyurethane and/or polyisocyanurate based polymers. Stabilizing/flow additives (e.g. one to three hydrocarbon-based liquids) may be in the range of about 0% to about 3% weight of the polymer of the foam composition or of the cushion. The stabilizing additives may be used to improve flow and/or the wetting properties of polyurethane and/or polyisocyanurate-based polymers. Auxiliary agents, such as liquid CO₂, may be used to soften the foam and gel catalysts (e.g. tin octoate) may be used to catalyze the reaction of isocyanate with the polyol. Gelation catalysts (e.g. one to four amine and/or quaternary salt-containing liquids) may be in the range of about 0% to about 6% weight of the polymer of the foam composition or of the cushion. The gelation catalysts may be used to catalyze polyurethane and/or polyisocyanurate reactions.

The foam composition and/or cushion may also include at least 40% filler (i.e. suspended solids) suspended in the polymer. For example, the filler may be or include an inorganic filler (e.g., calcium carbonate, silica, fly ash, barium sulfate, etc.) and/or an organic filler (e.g., hydrocarbon gels, phase change materials, etc.). Various embodiments of the filler may additionally, or alternatively, include a filler that is electrically conductive and/or is, or can be, magnetized such as, for example, metallic materials.

The foam composition and/or cushion includes a plurality of digesters suspended in the polymer. The filler itself may consist of the plurality of digesters, according to one embodiment, or the plurality of digesters may be included in addition to the filler, according to another embodiment. For instance, the filler may be used to reduce the amount of the polymer in the foam, and the digesters may also be added in addition to the filler.

According to one embodiment, the digesters may be dormant for a period of time, and then may be activated during conditions that would be typical during customary disposal of the foam and/or cushion. For instance, the digesters may be encapsulated such that the digesters are inactive until such time that the encapsulation breaks or is otherwise breached. Upon breach of the encapsulation, the digesters may be activated. In other embodiments, the digesters may not require individual encapsulation and may be suspended in the polymer. Specifically, the digesters may be selected based on the digesters' ability to break urethane bonds, or otherwise cause the polymer to degrade or decompose. The encapsulation may be breached due to, for example, typical environmental conditions where the foam or cushion would be discarded (e.g., a landfill). Such environmental conditions that might breach the encapsulation can include water, light, soil, etc.

In various embodiments, the polymer itself may surround the digesters such that the digesters are suspended in the polymer. The digesters will remain inactive until exposed to water. Upon customary disposal of the foam or cushion, water may penetrate into the pores of the polymer and the digesters may become activated. The water would cause the digesters to transport enzymes out and transport metabolites in, and the digesters grow/proliferate. This continual process will cause the polymer (e.g. the urethane bonds of the polymer) to gradually break down as long as the polymer is exposed to water.

Further, according to one embodiment, if water were temporarily applied to the foam or cushion, or the foam or cushion were to be washed (e.g. in a washing machine) or cleaned, although the water under those conditions could begin the degradation process, once the foam or pillow became dry the possible degradation process would stop. Thus, while there may be small levels of degradation due to a single washing or cleaning of the foam or cushion, the exposure to water during customary use of the foam or cushion would which would limit the amount of polymer breakdown. Continual washings or cleanings may eventually lead to advanced levels of degradation of the polymer of the foam or cushion, but limited washings or cleanings should not dramatically affect the lifespan of the overall foam or cushion structure or functionality.

According to another embodiment, the outer surface of the polymer may have an encapsulating barrier that protects the foam or cushion and prevents activation of the digesters. In particular, after customary disposal, the barrier on the outer surface may begin to decompose after a prolonged period of exposure to water, light, soil, etc., at which point the outer barrier on the outer surface may become breached and the digesters within the polymer would become activated. Once activated, the digesters may begin to degrade the polymer. The barrier may be or include a barrier fabric or other material, foam layer, spray coating, etc.

Various other methods and processes for encapsulating or otherwise inhibiting activation of the digesters during customary use of the foam or cushion are also contemplated herein.

Further, the filler may have a percent weight within the range of about 40% to about 95%, within the range of about 45% to about 95%, within the range of about 50% to about 95%, within the range of about 55% to about 95%, within the range of about 60% to about 95%, within the range of about 65% to about 95%, within the range of about 70% to about 95%, within the range of about 75% to about 95%, within the range of about 80% to about 95%, within the range of about 82% to about 90%, or within the range of about 90% to about 95%. In a particular embodiment, the disclosed foam composition and/or pillow may include about 85% filler.

In order to determine how to produce a foam composition or a cushion that includes foam where at least 40% filler suspended in the polyurethane, the amount of foam to be produced for a given mold, e.g. an aluminum mold, needs to be calculated. Initially, given the mold volume, B, and the nominal density of the foam, A, the amount of foam needed to fill the void of the mold is determined using the following equation:

$$\frac{A \times B}{0.2625 grams / {in}^{3}} .$$

The nominal density of the foam, A, may be expressed in pounds per cubic foot, and the mold volume, B, may be expressed in cubic inches. After multiplying the nominal density by the mold volume, the product may be divided by the conversion factor of 0.2625, which is expressed in grams per cubic inch.

Based on the desired percentage of the filler (e.g., organic filler or inorganic filler) the lift capacity of the foam once the blowing agent is added is calculated at standard atmospheric pressure. When combined with the pressure difference across the mold volume, the amount of vacuum pressure needed to offset inefficiency of the foam lift can be estimated using the following equation:

$$\frac{C}{F} ,$$

where C′ is the mass of the filler, and

$$F = \frac{D \times E}{D + E}$$

where D is the density of the foam and E is the density of the inorganic filler. Further, the capacity of a foam reaction to do non-mechanical work and release heat may be calculated using the following change in enthalpy equation: ΔHvap=ΔUvap+pΔV where ΔHvap is the enthalpy of vaporization, ΔUvap is the change in internal energy of the vapor, p is the pressure, and ΔV is the change in volume.

Foam compositions that do not include filler/suspended solids may include the following parts per hundred polyol (pphp): 100 polyol, 0.8 flow/stabilizing additive, 1.0 surfactant, 0.10 blow catalyst, 0.55 gelation catalyst, 3.40 blowing agent and 64.97 isocyanate. The total pphp would be 170.82. Further, based on an isocyanate index of 100, the density in pounds per cubic foot (pcf) would be 2.785 and 50% indention force deflection (IFD) would be approximately 79 pounds per square inch (psi). When calculated in parts by weight (pbw) the composition may include: 58.55 polyol, 0.47 flow/stabilizing additive, 0.59 surfactant, 0.06 blow catalyst, 0.32 gelation catalyst, 1.99 blowing agent, and 38.03 isocyanate. The total pbw would then be 100.00.

Further, a foam composition having the following batch wet weight of 819.59 polyol, 6.56 flow/stabilizing additive, 8.2 surfactant, 0.82 blow catalyst, 4.51 gelation catalyst, 27.87 blowing agent, and 532.46 isocyanate, leads to a total batch wet weight of 1,400.00 grams. The isocyanate index for this composition would be 100 and the approximate density may be approximately 2.4 pcf. This would result in an approximate weight of 1,050-1,100 grams, and more particularly approximately 1,075 grams.

The following non-limiting example of a foam composition that includes 55% filler/suspended solids may be used. In particular, foam composition may include a batch wet weight of 468.36 polyol, 3.75 flow/stabilizing additive, 4.68 surfactant, 0.47 blow catalyst, 2.58 gelation catalyst, 15.92 blowing agent, 600 filler (e.g., inorganic filler), and 304.24 isocyanate. The total batch wet weight would be 1,400.00 grams, which would correspond to a foam having a length of 584.20 mm, a width of 398.78 mm and a height of 120 mm. The isocyanate index would be 100 and the density would be approximately 2.61 pcf. This would result in an approximate weight of about 1,168 grams.

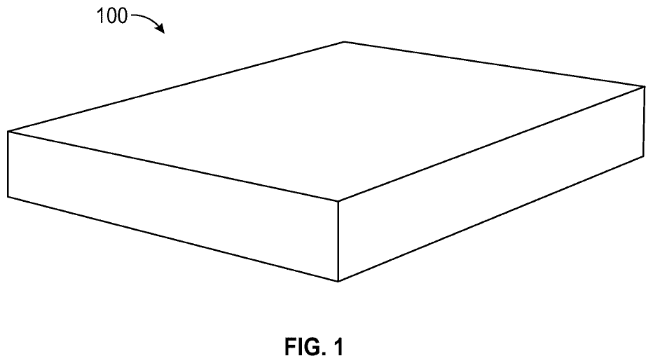

Referring now to FIG. 1, an exemplary foam slab 100 is illustrated. The foam slab 100 may include a foam compositing having a polymer and at least 40% filler suspended in the polymer. Further, the polymer includes a maximum of 60% polyol, which includes multiple hydroxyl groups, and less than 50% isocyanate. In various embodiments, the polymer may be a polyurethane or a polyisocyanurate-based polymer. Further, the polymer may include a polyol within the range of about 2% to about 60%, within the range of about 2 to about 55%, within the range of about 2% to about 50%, within the range of about 2% to about 45%, within the range of about 2% to about 40%, within the range of about 2% to about 35%, within the range of about 2% to about 30%, within the range of about 2% to about 25%, within the range of about 2% to about 20%, within the range of about 2% to about 15%, within the range of about 2% to about 10%, or within the range of about 2% to about 8%. Example polyols include petroleum, natural oils, animal by-products, vegetable oil, etc.

Further, the polymer of the foam composition of the foam slab 100 may include isocyanate with an amount of less than 50% isocyanate. In various embodiments, the isocyanate may be within the range of about 1% to about 50%, within the range of about 1% to about 45%, within the range of about 1% to about 40%, within the range of about 1% to about 35%, within the range of about 1% to about 30%, within the range of about 1% to about 25%, within the range of about 1% to about 20%, within the range of about 1% to about 15%, within the range of about 1% to about 10%, or within the range of about 1% to about 5%. Example isocyanate include methylene isocyanate-based liquid or toluene isocyanate-based liquid. The foam slab 100 may also include at least 40% filler (i.e. suspended solids) suspended in the polymer, such as the inorganic filler or organic filler discussed above. Further, various embodiments of the foam slab 100 may include various percentages of filler, such as the example percentages disclosed above. In one particular embodiment, the foam slab 100 may include 85% filler.

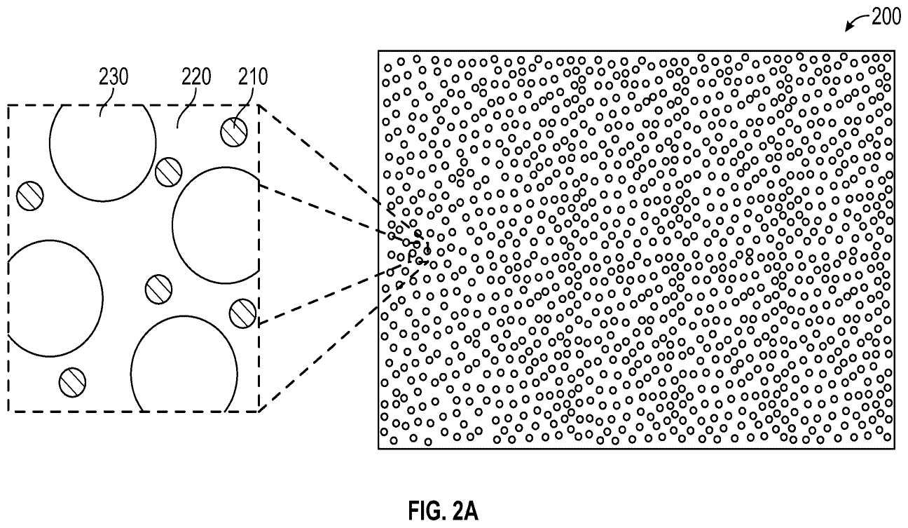

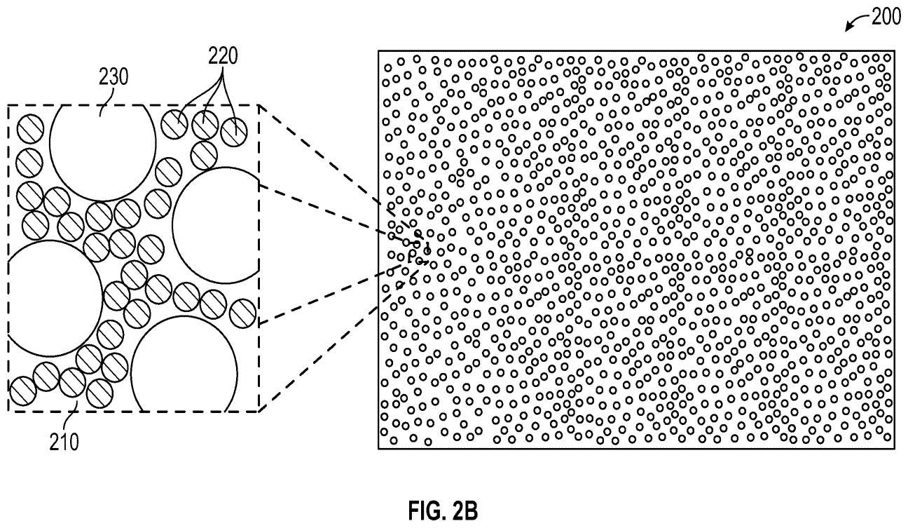

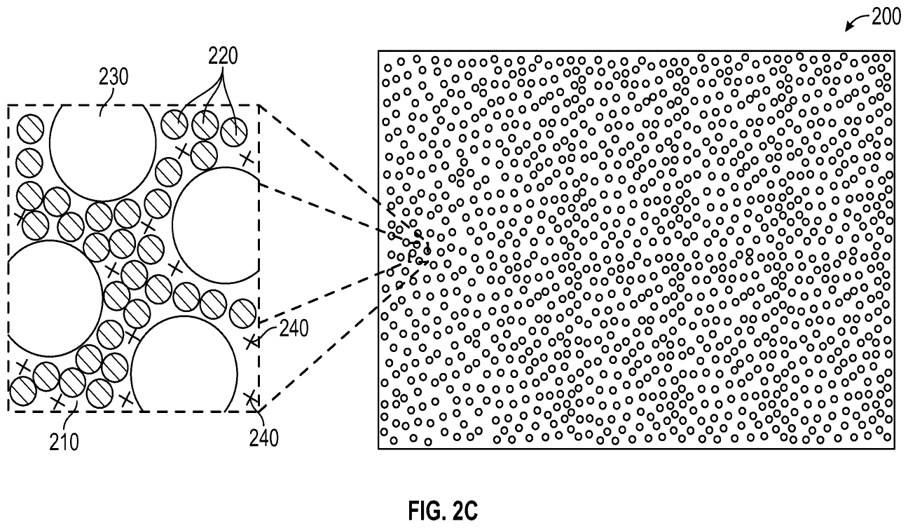

FIG. 2A depicts a magnified side view of an exemplary foam slab 200. The foam slab 200 may include a polymer 210, within which a plurality of fillers 220 are suspended, that surrounds a plurality of pores 230. In particular, although not necessarily drawn to scale and as depicted in FIG. 2A, the fillers 220 may be approximately 40%. In contrast, referring now to FIG. 2B, other embodiments may include as much as 95% fillers 220, and more particularly 85% filler 220.

Referring now to FIG. 2C, a magnified side view of the exemplary foam slab 200 of FIG. 2B that further includes a plurality of digesters 240 suspended in the polymer 210. According to one embodiment, the digesters 240 may be separate from the filler 220.

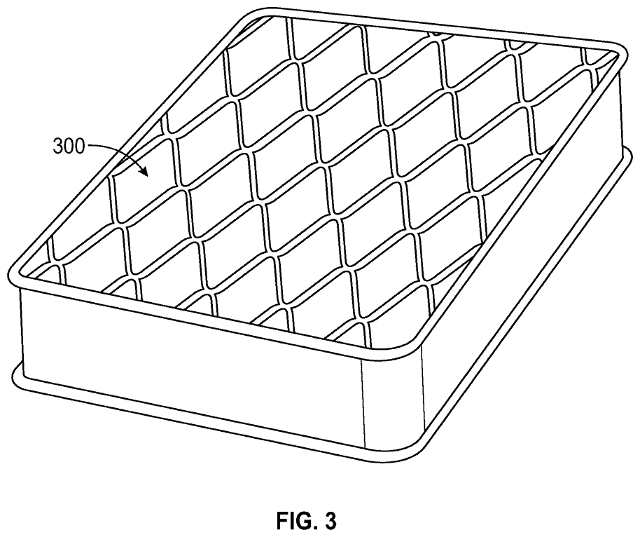

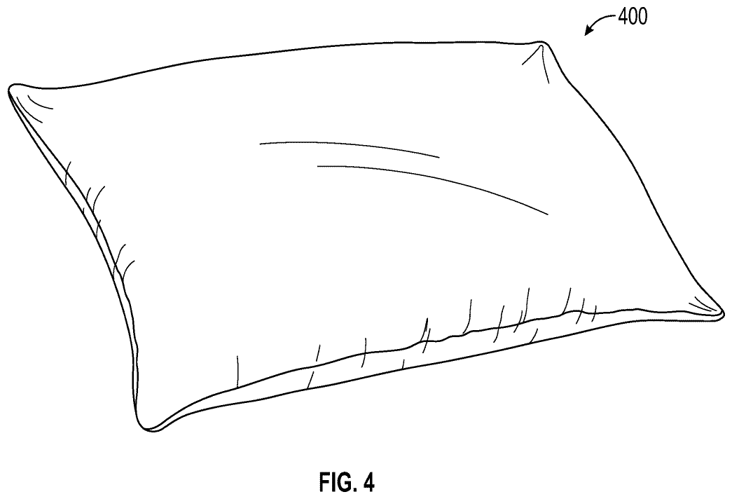

Example cushions that include a polymer and at least 40% filler suspended in the polymer are depicted in FIGS. 3-4. In particular, a mattress 300 or a pillow 400 may include a polymer and at least 40% filler suspended in the polymer.

Not only does increasing the percentage of filler suspended in the polymer reduce the amount of polymer (e.g. polyurethane), which may provide improvements over existing foam compositions during disposal, and the digesters will catalyze the breakdown of the polymer during exposure to water, another unexpected advantage and improvement over current foams used in cushions is that the filler (i.e. suspended solid) or digester can have improved resistance to flammability. Typically, foam mattresses are required to use a flame resistant/retardant (FR) fire barrier fabric/sock/cap that may be treated with fire-resistant chemicals or commonly include fiberglass fabric. Most often, fiberglass FR fire barriers are used so that the mattresses can satisfy federally mandated fire tests. By increasing the percentage of filler or suspended solid and/or the digesters in the polymer, cushions (i.e., mattresses) can satisfy the requirements of federally mandated fire tests without the need of a FR fire barrier, which advantageously eliminates the need to incorporate fiberglass FR fire barriers. Fiberglass FR fire barriers are virtually indestructible and do not readily decompose after typical disposal. By eliminating the need for fiberglass FR fire barriers, additional environmental benefits are provided, which further catalyzes the degradation process of certain cushions (i.e., mattresses).

Also disclosed herein is a method of manufacturing that includes producing a foam composition or cushion that includes a polymer and at least 40% filler suspended in the polymer. Further, the polymer includes a maximum of 60% polyol, which includes multiple hydroxyl groups, and less than 50% isocyanate.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has” and “having”), “include” (and any form of include, such as “includes” and “including”), “contain” (and any form contain, such as “contains” and “containing”), and any other grammatical variant thereof, are open-ended linking verbs. As a result, a method or article that “comprises”, “has”, “includes” or “contains” one or more steps or elements possesses those one or more steps or elements, but is not limited to possessing only those one or more steps or elements. Likewise, a step of a method or an element of an article that “comprises”, “has”, “includes” or “contains” one or more features possesses those one or more features, but is not limited to possessing only those one or more features.

As used herein, the terms “comprising,” “has,” “including,” “containing,” and other grammatical variants thereof encompass the terms “consisting of” and “consisting essentially of.”

The phrase “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed compositions or methods.

All publications cited in this specification are herein incorporated by reference as if each individual publication were specifically and individually indicated to be incorporated by reference herein as though fully set forth.

Subject matter incorporated by reference is not considered to be an alternative to any claim limitations, unless otherwise explicitly indicated.

Where one or more ranges are referred to throughout this specification, each range is intended to be a shorthand format for presenting information, where the range is understood to encompass each discrete point within the range as if the same were fully set forth herein.

While several aspects and embodiments of the present invention have been described and depicted herein, alternative aspects and embodiments may be affected by those skilled in the art to accomplish the same objectives. Accordingly, this disclosure and the appended claims are intended to cover all such further and alternative aspects and embodiments as fall within the true spirit and scope of the invention.

What is claimed is:

1. A cushioning foam composition, comprising:
a polymer comprising:
a maximum of 60% polyol comprising multiple hydroxyl groups;
less than 50% isocyanate;
at least 40% filler suspended in the polymer; and
a plurality of digesters suspended in the polymer.

2. The cushioning foam composition of claim 1, wherein the polymer comprises at least one of polyurethane and polyisocyanurate.

3. The cushioning foam composition of claim 1, wherein the polymer comprises polyol with an amount within the range of about 2% to about 60%.

4. The cushioning foam composition of claim 1, wherein the polymer comprises polyol with an amount within the range of about 2% to about 8%.

5. The cushioning foam composition of claim 4, wherein the polyol comprises at least one of petroleum, natural oil and animal by-products.

6. The cushioning foam composition of claim 1, wherein the polymer comprises isocyanate with an amount within the range of about 1% to about 50%.

7. The cushioning foam composition of claim 1, wherein the polymer comprises isocyanate with an amount within the range of about 1% to about 5%.

8. The cushioning foam composition of claim 7, wherein the isocyanate comprises at least one of methylene isocyanate based liquid and toluene isocyanate based liquid.

9. The cushioning foam composition of claim 1, further comprising at least one of a stabilizing additive, a surfactant, a blow catalyst, a blowing agent and a gelation catalyst.

10. The cushioning foam composition of claim 1, wherein the filler amount is within the range of about 85%-95% of the polymer.

11. The cushioning foam composition of claim 1, wherein the filler amount is within the range of about 90%-95% of the polymer.

12. The cushioning foam composition of claim 1, wherein the filler amount is at least about 75% of the polymer.

13. The cushioning foam composition of claim 12, wherein the filler comprises at least one of calcium carbonate, silica, fly ash, barium sulfate, hydrocarbon gels, phase change materials, electrically conductive material, magnetizable material.

14. The cushioning foam composition of claim 12, wherein the filler comprises the plurality of digesters.

15. The cushioning foam composition of claim 12, wherein the filler consists of the plurality of digesters.

16. The cushioning foam composition of claim 12, wherein the plurality of digesters comprise at least one of algae, microbes and fungus.

17. The cushioning foam composition of claim 12, wherein the plurality of digesters are individually encapsulated.

18. The cushioning foam composition of claim 1, wherein a barrier encapsulates the polymer.

19. A cushion, comprising:
a cushioning portion formed of the cushioning foam composition of claim 1.

20. A method of manufacturing a cushioning foam composition, comprising:
forming a polymer comprising:
a maximum of 60% polyol comprising multiple hydroxyl groups;
less than 50% isocyanate;
at least 40% filler suspended in the polymer; and
a plurality of digesters suspended in the polymer.
